(19) United States
(12) Patent Application Publication
(10) Pub. No.: US 2016/0243015 A1
(43) Pub. Date: Aug. 25, 2016

(54) SKIN LIGHTENING COMPOSITION

(71) Applicant: Conopco, Inc., d/b/a UNILEVER, Englewood Cliffs, NJ (US)

(72) Inventors: Martin Richard GREEN, Cambridge (GB); Rebecca Susan GINGER, Mears Ashby, Northamptonshire (GB); Robert Charles GLEN, Upend, Suffolk (GB)

(73) Assignee: Conopco, Inc., d/b/a UNILEVER, Englewood Cliffs, NJ (US)

(21) Appl. No.: 15/030,438

(22) PCT Filed: Oct. 14, 2014
(86) PCT No.: PCT/EP2014/072000

§ 371 Date: Apr. 19, 2016

(30) Foreign Application Priority Data
Oct. 25, 2013 (EP) 13190364.3

(51) Int. Cl. A61K 8/49; A61Q 15/00; A61K 8/34; A61Q 19/02; A61Q 17/04

(52) U.S. Cl. CPC A61K 8/4926; A61Q 19/02; A61Q 17/04; A61K 8/347; A61Q 15/00

(57) ABSTRACT

Desired skin colour is a major unmet consumer need around the world and especially in Asia. Consumers particularly desire even skin colour, absence of age spots (solar lentigines), absence of hyperpigmentation and lighter overall skin tone. One solution is to use biological actives that reduce the activity of melanocyte cells in skin. These cells, present in the basal layer of the epidermis, make the dark coloured pigment melanin and export it, in small export vesicles called melanosomes, to the neighbouring keratinocytes. It is well described in the literature that compounds which reduce melanin synthesis when topically applied to the skin will reduce skin darkness over time and can generate a more even skin tone. Tyrosinase is a very popular target for the regulation of melanocyte pigment production. However effective inhibitors of tyrosinase are bedevilled by safety issues causing, for example, melanocyte cell death, permanent depigmentation, irritation and allergic reactions. Often effective inhibitors kill melanocytes (for example hydroquinone) or cause sensitisation reactions. There is therefore a great need for safe and effective inhibitors of skin pigment production that work through an alternative safe mechanism. The inventors have observed that selected compounds of the same generic structure: or a salt thereof; wherein R₁, R₂, R₃, R₄ and R₅ may be independently selected from the group consisting of —H, -halide, and methyl, ethyl, propyl, iso-propyl, butyl, and t-butyl moieties, inhibit melanin production in Melanoderms™.

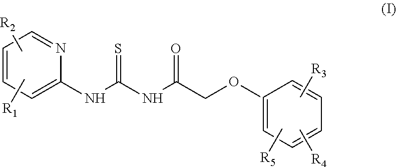

This invention relates to a topical skin lightening composition, in particular to a topical skin lightening composition comprising:

(a) A compound of formula (I):

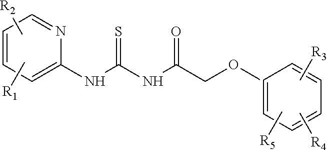

or a salt thereof;
wherein R₁, R₂, R₃, R₄ and R₅ may be independently selected from the group consisting of —H, -halide, and methyl, ethyl, propyl, iso-propyl, butyl, and t-butyl moieties; and

(b) A dermatogically acceptable vehicle.

Desired skin colour is a major unmet consumer need around the world and especially in Asia. Consumers particularly desire even skin colour, absence of age spots (solar lentigines), absence of hyperpigmentation and lighter overall skin tone. One solution is to use biological actives that reduce the activity of melanocyte cells in skin. These cells, present in the basal layer of the epidermis, make the dark coloured pigment melanin and export it, in small export vesicles called melanosomes, to the neighbouring keratinocytes. It is well described in the literature that compounds which reduce melanin synthesis when topically applied to the skin will reduce skin darkness over time and can generate a more even skin tone.

Tyrosinase is a very popular target for the regulation of melanocyte pigment production. However effective inhibitors of tyrosinase are bedevilled by safety issues causing, for example, melanocyte cell death, permanent depigmentation, irritation and allergic reactions. Often effective inhibitors kill melanocytes (for example hydroquinone) or cause sensitisation reactions. There is therefore a great need for safe and effective inhibitors of skin pigment production that work through an alternative safe mechanism.

Thanigaimalai et al (Bioorganic & Medicinal Chem. Letters, 21, 6824-6828 (2011)) describe a study the effect of a series of 1-phenylthioureas and 1,3-disubstituted thioureas against melanin formation in melanoma B16 cell line and mushroom tyrosinase. Inhibitory activity of tyrosinase of the 1-phenylthioureas is parallel to their melanogenic inhibition. Thus, the melanogenic inhibition in melanoma B16 cells of the 1-phenylthioureas could be the result of inhibition of tyrosinase. However the 1,3-disubstituted thioureas appear as melanogenic inhibitors without inhibition of tyrosinase.

CN 19 03 203 (Fudan University) discloses use of a polysubstituted acyl thiouracil derivative in preparing an anti-viral medicine.

US 2006/0135618 (Jean et al) discloses a medicine or a cosmetic composition comprising at least one generic thiourea, or at least one of its monoxide or dioxide derivatives, or mixtures thereof. The medicine is advantageously used for inhibiting tyrosinase, and as an anti-mutagenic and anti-carcinogenic agent.

US 2002/0044914 (Dooley et al) discloses methods and formulations for reducing pigmentation in skin using an array of compounds selected from benimidazoles, phenylthioureas, phenylthiols, phenylamines, bi-and multicyclic phenols, thiopheneamines and benzothiamides.

JP 56 32460 (Nihon Tokushu Noyaku Seizo) discloses acyl pyridyl thiourea derivatives of generic formula which are useful in controlling certain pathogenic fungi.

Liu et al (Chem. Res. Chinese Universities, 26(6), 929-932 (2010)) discloses synthesis of a series of compounds derived from 2,4-dichlorophenoxyacetyl(thio)urea and S-(+)-3-methyl-2-(4-chlorophenyl)butyramide and their subsequent testing for herbicidal and fungicidal activity.

The inventors have observed that selected compounds of the same generic structure are able to effectively reduce melanin in human cell living skin equivalents containing keratinocytes and melanocytes (so called Melanoderms™). Melanoderms™ are in vitro 3D living skin equivalents containing both human keratinocytes and melanocytes and are used as a close mimic of the effect of compounds on human skin function in vivo. Furthermore the mechanism by which the selected compounds reduced melanin was not through tyrosinase inhibition.

Accordingly the aforementioned generic structure describes compounds which inhibit pigment production and which can be used to beneficially alter skin colour and treat hyperpigmentation disorders in a safe and effective manner.

### SUMMARY OF THE INVENTION

In a first aspect of the invention, a topical skin lightening composition is provided, the topical skin lightening composition comprising:

(a) A compound of formula (I):

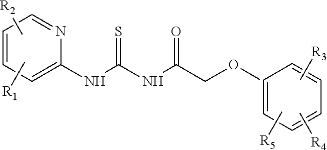

or a salt thereof;
wherein R₁, R₂, R₃, R₄ and R₅ may be independently selected from the group consisting of —H, -halide, and methyl, ethyl, propyl, iso-propyl, butyl, and t-butyl moieties; and

(b) A dermatogically acceptable vehicle.

In a second aspect of the invention, a method of skin lightening is provided, the method comprising the step of applying to skin the topical skin lightening composition of the first aspect of the invention.

In a third aspect of the invention, a method of skin lightening is provided, the method comprising the step of applying to skin a compound of formula (I):

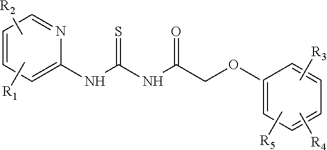

or a salt thereof;

wherein R₁, R₂, R₃, R₄ and R₅ may be independently selected from the group consisting of —H, -halide, and methyl, ethyl, propyl, iso-propyl, butyl, and t-butyl moieties.

### DETAILED DESCRIPTION OF THE INVENTION

In a first aspect of the invention, a topical skin lightening composition is provided, the topical skin lightening composition comprising:

(a) A compound of formula (I):

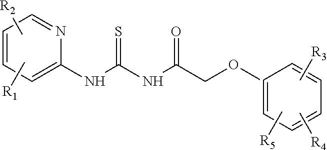

or a salt thereof;
wherein R₁, R₂, R₃, R₄ and R₅ may be independently selected from the group consisting of —H, -halide, and methyl, ethyl, propyl, iso-propyl, butyl, and t-butyl moieties; and

(b) A dermatogically acceptable vehicle.

In one embodiment, R₁ is —I or —Cl, and R₂ is H or a methyl moiety. In this embodiment, R₁ and R₂ are preferably at positions 5 and 6 on the pyridinyl moiety respectively.

R₃, R₄ and R₅ can be —H.

In one embodiment, R₃ and R₄ can be H. In this embodiment, R₅ is preferably at the ortho or para position on the phenyl moiety.

The topical skin lightening composition can comprise 5 to 0.0005, preferably 2 to 0.005, most preferably 0.5 to 0.05% w/w compound of formula (I).

The topical skin lightening composition can further comprise an ingredient selected from the group consisting of a fragrance, an additional skin lightening compound, a surfactant, an organic sunscreen, an inorganic sunscreen, an extender pigment, a preservative, an antiperspirant active, and a deodorant active.

The additional skin lightening compound can be selected from the group consisting of niacinamide, a resorcinol, koijic acid, retinol, retinyl esters including retinyl palmitate, 12-hydroxystearic acid, lactic acid, ascorbic acid, glabradin, dioic acids including octadecenedioic acid and azeleic acid, resveratrol, ascorbyl phosphate, ascorbyl palmitate, acetyl glucosamine, calcium pantothenate, alpha arbutin, climbazole, pitera extract, soybean extract, undecylenoyl phenylalanine, aqueous extract of bearberry and mixtures thereof.

The resorcinol can be selected from the group consisting of 4-ethyl resorcinol, 4-hexyl resorcinol and phenylethyl resorcinol.

In one embodiment, the additional skin lightening compound is a non-tyrosinase inhibiting skin lightening compound.

Typically, the topical skin lightening composition can comprise 0.0001-10, preferably 0.01-2, most preferably 0.1-1% w/w of additional skin lightening compound.

The topical skin lightening composition preferably comprises a sunscreen to protect the skin against UV-A and/or UV-B solar radiation. Sunscreens include those materials commonly employed to block ultraviolet light. Illustrative organic compounds are the derivatives of p-aminobenzoic acid (PABA), cinnamate and salicylate. For example, avobenzophenone (Parsol 1789®), octyl methoxycinnamate and 2-hydroxy-4-methoxy benzophenone (also known as oxybenzone) can be used. Octyl methoxycinnamate and 2-hydroxy-4-methoxy benzophenone are commercially available under the trade marks, Parsol MCX and Benzophenone-3, respectively. Ecamsule, a benzylidene camphor derivative, sold under the trade mark Mexoryl SX, and drometrizole trisiloxane, a benzotriazole sold under the trade mark Mexoryl XL, may also be used. Still other examples include octocrylene, phenylbenzimidazole sulfonic acid (also known as ensulizole), ethylhexyl salicylate,diethylhexyl naphthylate, bimotrizinole (trade marked as Tinosorb S) and bisoctrizole (Tinosorb M).

Inorganic sunscreens include oxides like titanium dioxide and zinc oxide which reflect or scatter the suns rays. The exact amount of sunscreen employed in the compositions can vary depending upon the degree of protection desired from the sun's UV radiation.

Typically, the topical skin lightening composition comprises 0.01 to 15, preferably 0.1 to 10, most preferably 0.5 to 7.5% w/w an inorganic sunscreen and/or organic sunscreen.

The topical skin lightening composition preferably comprises an anti-perspirant active. Anti-perspirant actives are preferably incorporated in an amount of from 0.5 to 50, particularly from 5 to 30 and especially from 10 to 26% by weight of the topical skin lightening composition, including all ranges subsumed therein.

Anti-perspirant actives for use herein are often selected from astringent active salts, including in particular aluminium, zirconium and mixed aluminium/zirconium salts, including both inorganic salts, salts with organic anions and complexes. Preferred astringent salts include aluminium, zirconium and aluminium/zirconium halides and halohydrate salts, such as chlorohydrates.

Deodorant actives include particularly bactericides, such as chlorinated aromatics, including biguanide derivatives, of which triclosan (e.g. Irgasan DP300 or Triclorban) and chlorhexidine warrant specific mention. Another class of effective deodorant active comprises polyaminopropyl biguanide salts such as are available under the trade mark Cosmosil. Such materials commonly act as bactericides. A still further suitable class of materials comprise chelators that can sequester iron, and thereby retard bacterial growth, including aminopolycarboxylates such as ethylenediamine tetraacetic acid (EDTA) or higher homologues such as diethylenetriamine pentaacetic acid (DTPA). Deodorant actives other than astringent metal antiperspirant salts are commonly employed at a concentration of from 0.1 to 5, and particularly 0.1 to 2% by weight of the topical skin lightening composition, including all ranges subsumed therein.

Fragrances may be used in the topical skin lightening composition. Illustrative non-limiting examples of the types of fragrances that may be used include those comprising terpenes and terpene derivatives like those described in Bauer, K., et al., Common Fragrance and Flavor Materials, VCH Publishers (1990).

Illustrative yet non-limiting examples of the types of fragrances that may be used in this invention include myrcene, dihydromyrenol, citral, tagetone, cis-geranic acid, citronellic acid, mixtures thereof or the like.

Preferably, the amount of fragrance employed in the topical skin lightening composition is in the range from 0.0 to 10, more preferably 0.00001 to 5, most preferably 0.0001 to 2% by weight of the topical skin lightening composition, including all ranges subsumed therein.

The dermatologically acceptable carrier for the topical skin lightening composition act as diluents, dispersants and/or carriers for the compound and for any other optional but often preferred ingredients. The carrier may be aqueous-based, anhydrous or an emulsion whereby a water-in-oil or oil-in-water emulsion is generally preferred. If the use of water is desired, water typically makes up the balance of the topical skin lightening composition of the second aspect of the invention, and preferably makes up from 5 to 98%, and most preferably from 40 to 80% by weight of the topical skin lightening composition, including all ranges subsumed therein.

In addition to water, organic solvents may be optionally included to act as carriers or to assist carriers within the compositions of the present invention. Illustrative and non-limiting examples of the types of organic solvents suitable for use in the present invention include alkanols like ethyl and isopropyl alcohol, mixtures thereof or the like.

Other suitable organic solvents include ester oils like isopropyl myristate, cetyl myristate, 2-octyldodecyl myristate, avocado oil, almond oil, olive oil, neopentylglycol dicaprate, mixtures thereof or the like. Typically, such ester oils assist in emulsifying the composition of this invention, and an effective amount is often used to yield a stable, and most preferably, water-in-oil emulsion.

Emollients may also be used, if desired, as carriers within the topical skin lightening composition. Alcohols like 1-hexadecanol (i.e. cetyl alcohol) are often desired as are the emollients generally classified as silicone oils and synthetic esters. Silicone oils suitable for use include cyclic or linear polydimethylsiloxanes containing from 3 to 9, preferably from 4 to 5, silicon atoms. Non-volatile silicone oils useful as an emollient material in the inventive composition described herein include polyalkyl siloxanes, polyalkylaryl siloxanes and polyether siloxane copolymers. The essentially non-volatile polyalkyl siloxanes useful herein include, for example, polydimethylsiloxanes. Silicone elastomers may also be used.

The ester emollients that may optionally be used are:
(1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.
(2) Ether-esters such as fatty acid esters of ethoxylated fatty alcohols.
(3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) Wax esters such as beeswax, spermaceti, stearyl stearate and arachidyl behenate.
(5) Sterols esters, of which cholesterol fatty acid esters are examples.

Emollients, when used, typically make up from 0.1 to 50% by weight of the topical skin lightening composition, including all ranges subsumed therein.

Fatty acids having from 10 to 30 carbon atoms may also be included as acceptable carriers within the composition of the present invention. Illustrative examples of such fatty acids include pelargonic, lauric, myristic, palmitic, stearic, isostearic, oleic, linoleic, arachidic, behenic or erucic acid, and mixtures thereof. Compounds that are believed to enhance skin penetration, like dimethyl sulfoxide, fatty acids and ethanol may also be used as an optional carrier.

Humectants of the polyhydric alcohol type may also be employed in the topical skin lightening compositions. The humectant often aids in increasing the effectiveness of the emollient, reduces scaling at the skin surface, stimulates removal of built-up scale and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably propylene glycol or sodium hyaluronate. Other humectants which may be used include hydroxyethyl urea. The amount of humectant may range anywhere from 0.2 to 25%, and preferably, from 0.5 to 15% by weight of the topical skin lightening composition, including all ranges subsumed therein.

Moisturisation may be improved through use of petrolatum or paraffins.

Thickeners may also be utilized as part of the acceptable carrier in the topical skin lightening compositions. Typical thickeners include cross-linked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Amounts of the thickener may range from 0.0 to 5, usually from 0.001 to 1, optimally from 0.01 to 0.5% by weight of the topical skin lightening composition, including all ranges subsumed therein.

Collectively the water, solvents, silicones, esters oils, emollients, fatty acids, humectants and/or thickeners will constitute the acceptable carrier in amounts from 1 to 99.9, preferably from 80 to 99% by weight of the topical skin lightening composition.

Surfactants may also be present in topical skin lightening compositions. Total concentration of the surfactant will range from about 0 to about 40%, and preferably from about 0 to about 20%, optimally from about 0 to about 5% by weight of the topical skin lightening composition. The surfactant may be selected from the group consisting of anionic, nonionic, cationic and amphoteric actives. Particularly preferred nonionic surfactants are those with a C10-C20 fatty alcohol or acid hydrophobe condensed with from 2 to 100 moles of ethylene oxide or propylene oxide per mole of hydrophobe; mono- and di-fatty acid esters of ethylene glycol; fatty acid monoglyceride; sorbitan, mono- and di-C8-C20 fatty acids; block copolymers (ethylene oxide/propylene oxide); and polyoxyethylene sorbitan as well as combinations thereof. Alkyl polyglycosides and saccharide fatty amides (e.g. methyl gluconamides) are also suitable nonionic surfactants.

Preferred anionic surfactants include soap, alkyl ether sulfate and sulfonates, alkyl sulfates and sulfonates, alkylbenzene sulfonates, alkyl and dialkyl sulfosuccinates, C8-C20 acyl isethionates, acyl glutamates, C8-C20 alkyl ether phosphates and combinations thereof.

Various types of optional additional active ingredients may be used in the topical skin lightening compositions. Actives are defined as skin benefit agents other than emollients and other than ingredients that merely improve the physical characteristics of the composition. Although not limited to this category, general examples include extender pigments such as talcs and silicas, as well as alpha-hydroxy acids, beta-hydroxy acids and zinc salts.

Beta-hydroxy acids include salicylic acid, for example. Zinc oxide and zinc pyrithione are examples of zinc salts useful in the topical skin lightening composition.

Many compositions, especially those containing water, should be protected against the growth of potentially harmful microorganisms. Anti-microbial compounds, such as triclosan, and preservatives are, therefore, typically necessary. Suitable preservatives include alkyl esters of p-hydroxybenzoic acid, hydantoin derivatives, propionate salts, and a variety of quaternary ammonium compounds. Particularly preferred preservatives are methyl paraben, propyl paraben, phenoxyethanol and benzyl alcohol. Preservatives will usually be employed in amounts ranging from 0.1 to 2% by weight of the topical skin lightening composition.

Still other optional ingredients that may be used with the topical skin lightening composition include dioic acids (e.g. malonic acid and sebacic acid), antioxidants like vitamin E, retinoids, including retinoic acid, retinal, retinol and retinyl esters such as retinyl propionate and retinyl palmitate, conjugated linoleic acid, petroselinic acid and mixtures thereof, as well as any other conventional ingredients well known for wrinkle-reducing (such as hyaluronic acid, ubiquinone, jasmonic acid derivatives, collagen, peptides and proxylane), anti-acne effects and reducing the impact of sebum.

When making the topical skin lightening composition, the desired ingredients are mixed in no particular order and usually at temperatures from 70 to 80° C. and under atmospheric pressure.

The packaging for the topical skin lightening composition can be a patch, bottle, tube, roll-ball applicator, propellant driven aerosol device, squeeze container or lidded jar.

In a second aspect of the invention, a method of skin lightening is provided, the method comprising the step of applying to skin the topical skin lightening composition of the first aspect of the invention.

In a third aspect of the invention, a method of skin lightening is provided, the method comprising the step of applying to skin a compound of formula (I):

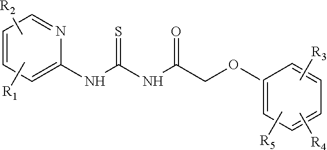

or a salt thereof;

wherein R₁, R₂, R₃, R₄ and R₅ may be independently selected from the group consisting of —H, -halide, and methyl, ethyl, propyl, iso-propyl, butyl, and t-butyl moieties.

Preferably skin lightening is any one of the benefits selected from the group consisting of treating melasma, treating post-inflammatory hyperpigmentation, treating age spots, treating dark spots and treating uneven skin tone.

### EXAMPLE

Materials:

N-[[(5-iodo-2-pyridinyl)-amino]-thioxomethyl]-2-phenoxyacetamide (Akos catalogue number 003197696 and hereinafter referred to as NC-475)

N-[[(5-chloro-2-pyridinyl)-amino]-thioxomethyl]-2-phenoxyacetamide (Ambinter catalogue number 8450325 and hereinafter referred to as S12)

N-[[(5-chloro-2-pyridinyl)-amino]-thioxomethyl]-2-(4-methylphenoxy)-acetamide (Akos catalogue number 002340673 and hereinafter referred to as S13)

N-[[2-pyridinylamino]-thioxomethyl]-2-phenoxyacetamide (Ambinter catalogue number 3111725 and hereinafter referred to as S14)

N-[[(5-iodo-2-pyridinyl)-amino]-thioxomethyl]-2-(4-chloro-3,5-dimethylphenoxy)-acetamide (Ambinter catalogue number 6570626 and hereinafter referred to as S15)

N-[[(5-chloro-2-pyridinyl)-amino]-thioxomethyl]-2-[4-(1-methylethyl)-phenoxy]-acetamide (Ambinter catalogue number 1863778 and hereinafter referred to as S16)

N-[[(5-iodo-2-pyridinyl)-amino]-thioxomethyl]-2-(2,4-dichlorophenoxy)-acetamide (Ambinter catalogue number 1863700 and hereinafter referred to as S17)

N-[[(5-iodo-2-pyridinyl)-amino]-thioxomethyl]-2-(4-chlorophenoxy)-acetamide (Ambinter catalogue number 1863697 and hereinafter referred to as S18)

N-[[(5-iodo-2-pyridinyl)-amino]-thioxomethyl]-2-[4-(1,1-dimethylethyl)phenoxy]-acetamide (Akos catalogue number 003197702 and hereinafter referred to as S19)

N-[[(5-iodo-2-pyridinyl)-amino]-thioxomethyl]-2-(3-methylphenoxy)-acetamide (Akos catalogue number 003197706 and hereinafter referred to as S20)

N-[[(5-iodo-6-methyl-2-pyridiny)lamino]-thioxomethyl]-2-phenoxyacetamide (Akos catalogue number 003983260 and hereinafter referred to as S21)

Method:

Assay of pigment production by Living Skin Equivalents (Melanoderms™)

So called reconstructed pigmented ‘skin’ cultures containing keratinocytes and melanocytes are commercially available and, for example, can be purchased from MatTek Corp, 200, Homer Avenue, Ashland, Mass., USA, under the trade name Melanoderm™. Melanoderms™ (catalogue reference MEL-300) containing melanocytes from black donor skin were maintained in EPI-100-NMM113 media following the manufacturer's instructions and using standard methods for the maintenance of cultured mammalian cells under aseptic conditions. The Melanoderm™ cultures were treated with test agents for a total of 14 days. During the 14 day culture period the melanocytes in the Melanoderm™ cultures synthesise melanin in specialised vesicular structures called melanosomes. The pigmented melanosomes are transferred to the adjacent keratinocytes and the transferred-melanosomes tend to accumulate over the nucleus of the keratinocytes. This process is the same as occurs in skin in vivo and accordingly the Melanoderm™ culture is considered to be a very good test ‘skin tissue’ to mimic the effect of the skin pigmentation process in vivo. A compound that leads to a reduction in melanin production in the Melanoderm™ culture over 14 days by 10% or more is considered to have a positive pigment reducing effect. It is important that the reduction in pigment occurs without substantive loss of cell viability such that the inhibitory effect of the test compound is due to its biological function rather than due to cell toxicity.

Culture media changes and re-dosing of compounds at selected concentrations took place every 2 or 3 days during the test period. Melanoderm™ cultures were also treated with control materials including the vehicle (dimethylsulphoxide (DMSO)) used to dissolve the test material and a positive control material known to lead to the synthesis of reduced amounts of melanin in the cultures. 4-ethylresorcinol was used as a positive control. After 14 days culture, transmitted-light microscopy images were captured for every culture using a Leica DM IL microscope at ×20 magnification.

The cell viability of the cultures was assessed using the so called WST-1 (Water Soluble Tetrazolium Salt-1) assay purchased from Roche Applied Science and following the manufacturer's instructions. The stable tetrazolium salt WST-1 is cleaved to a soluble formazan dye by a complex cellular mechanism that occurs primarily at the cell surface. This bioreduction is largely dependent on the glycolytic production of NADPH in viable cells. Therefore, the amount of soluble formazan dye formed in the culture medium directly correlates to the number of metabolically active cells in the culture. Cultures were incubated with WST-1 in the medium for 1 hour and production of formazan measured at 450 nm using a spectrophotometer plate reader, e.g. a BMG Labtech FLUOstar plate reader. Cultures were considered ‘viable’ if they generated greater than 80% of the control value of formazan product. Subsequently the culture was washed briefly in phosphate buffer solution (PBS) and total melanin and protein extracted together from each culture using 250 μl Solvable™ (Perkin Elmer product 6NE9100) reagent at 60° C. overnight as recommended by the manufacturer. The total protein content using a sample of the extracts was determined using the so called BCA (bicinchoninic acid) assay. The BCA assay may be purchased from Pierce Chemical Company and conducted following the manufacturer's instructions. Melanin was determined by measuring the absorbance at 485 nm, for example using a BMG Labtech FLUOstar plate reader. The amount of melanin extracted from each culture was determined by reference to a standard synthetic-melanin curve (Sigma Aldrich Chemical Company catalogue reference M8631).

Tyrosinase Inhibition Assay

Human neonatal darkly pigmented melanocytes lysate was used as a source of tyrosinase enzyme. The human melanocyte cells were purchased from Cascade Biologics (Code: C-202-5C) and cultured using standard methods. A confluent 25 cm² flask of melanocytes was washed with 2 ml of ice-cold PBS (phosphate buffered saline), followed by incubation with 1 ml of (0.025% w/v) Trypsin (0.01% w/v) EDTA (ethylenediamine tetraacetic acid) solution for 2 minutes at 37° C. to release the adherent cells. Trypsin was neutralized by the addition of 3 ml ‘neutralizer’ solution (sterile PBS solution containing 0.5% w/vnewborn bovine serum). The loosened cells were spun down at 1000 rpm for about 10 minutes and the supernatant discarded. The cell pellet was lysed with 0.1 ml cell membrane lysis buffer (20 mM HEPES (2-[4-(2-hydroxyethyl)piperazin-1-yl]ethanesulfonic acid), 0.4 M NaCl, 1% w/v NP-40 (nonyl phenoxypolyethoxylethanol), 5 μl of protease inhibitor cocktail (SIGMA Code P1860) and 9 μl of 100 mM PMSF (phenylmethylsulfonyl fluoride), pH 7.2). The resultant suspension was sonicated gently for a few seconds on ice using a Bandelin sonoplus probe sonicator. The resultant lysate was centrifuged at about 5,000×g at 4° C. for 10 minutes and the supernatant used as the source of human tyrosinase enzyme. The protein content in the lysate was measured by the Bradford reagent method (bovine serum albumin standard). Typically, 5-10 μg protein equivalent of the cell lysate extract was used for each assay.

The solution phase tyrosinase activity assay was initiated by mixing 50 μl of 50 mM potassium phosphate buffer (pH 6.8), 60 μl of 10 mM MBTH (3-Methyl-2-benzothiazolinonhydrazon hydrochloride hydrate) in 10 mM potassium phosphate buffer, 30 μl of 10 mM L-DOPA (L-3,4-dihydroxyphenylalanine) in 10 mM potassium phosphate buffer, 4 μl of melanocytic cell extract (about 2 μg/μl protein), and 6 μl of distilled water (to make up the volume to 150 μl), and incubated for about 1 hour at 37° C. The colour generating tyrosinase reaction was stopped with addition of equal volume of ice cold 10% w/v TCA (trichloroacetic acid) in 10 mM potassium phosphate buffer and centrifuged at about 300×g for 3 minutes at 4° C. The soluble supernatant was carefully separated from the pellet and kept at room temperature for about 5 minutes. Then 200 μl of the supernatant was transferred to a 96-well flat-bottom plate, and the OD (optical density) read in a TECAN plate-reader (540 nm transmission). To study tyrosinase activity in the presence of any inhibitor, all the reaction ingredients except for L-DOPA, and the tyrosinase inhibitor(s) at the desired concentration were pre-mixed for about 15 minutes. The colour generating reaction was initiated by adding L-DOPA. Incubations with the inhibitors excluded, the tyrosinase extract excluded, and with known tyrosinase inhibitors such as 4-ethyl resorcinol or kojic acid, were used as control measures. Incubations were conducted in triplicate.

The ‘control’ OD reading in the absence of an added tyrosinase inhibitor was normalised to 100%. A test compound at 10 μM that gave an inhibition of colour generation by more than 10% of the control value was considered to be a tyrosinase inhibitor.

Results:

Melanin levels in the Melanoderm™ assay in each culture were expressed as values normalised to the amount of protein in the cultures and compared to control vehicle treated cultures and are tabulated in Table 1. A reduction of greater than 10% in the protein normalised melanin content of the cultures without substantive loss of cell viability, was considered to be a successful and novel pigment reducing effect. The cultures from the WST-1 assay results for all the test compounds in Table 1 were considered viable, ie more than 85% cells in the culture were metabolically active compared to a vehicle control.

TABLE 1
Melanoderm ™ assay results for test compounds as % inhibition of melanin production compared
to vehicle control. Melanin production was normalized to protein levels as previously described.

| Test | | 10 μM (% |
|---|---|---|
| compound | Structure | inhibition) |
| NC-0475 | 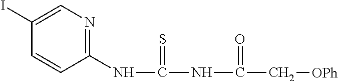 [SMILES: O=C(COc1ccccc1)NC(=S)Nc1ccc(I)cn1] | 53 |
| S12 | 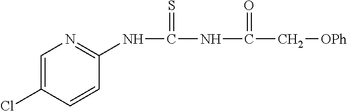 [SMILES: O=C(COc1ccccc1)NC(=S)Nc1ccc(Cl)cn1] | 47 |
| S13 | 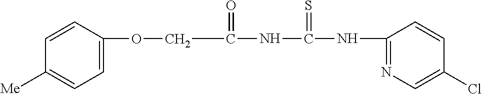 [SMILES: Cc1ccc(OCC(=O)NC(=S)Nc2ccc(Cl)cn2)cc1] | 35 |
| S14 | 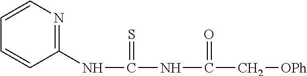 [SMILES: O=C(COc1ccccc1)NC(=S)Nc1ccccn1] | 10 |
| S15 | 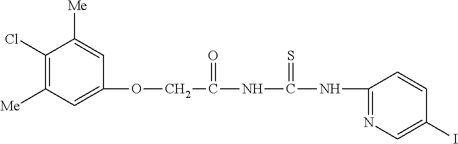 [SMILES: Cc1cc(OCC(=O)NC(=S)Nc2ccc(I)cn2)cc(C)c1Cl] | 43 |
| S16 | 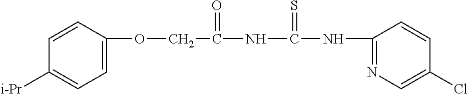 [SMILES: CC(C)c1ccc(OCC(=O)NC(=S)Nc2ccc(Cl)cn2)cc1] | 38 |
| S17 | 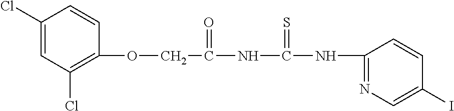 [SMILES: O=C(COc1ccc(Cl)cc1Cl)NC(=S)Nc1ccc(I)cn1] | 43 |
| S18 | 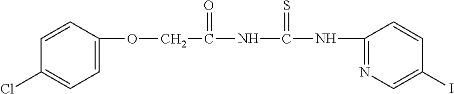 [SMILES: O=C(COc1ccc(Cl)cc1)NC(=S)Nc1ccc(I)cn1] | 28 |
| S19 | 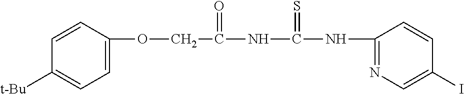 [SMILES: CC(C)(C)c1ccc(OCC(=O)NC(=S)Nc2ccc(I)cn2)cc1] | 22 |
| S20 | 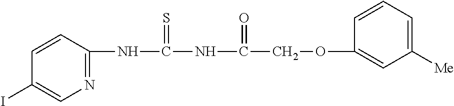 [SMILES: Cc1cccc(OCC(=O)NC(=S)Nc2ccc(I)cn2)c1] | 22 |
| S21 | 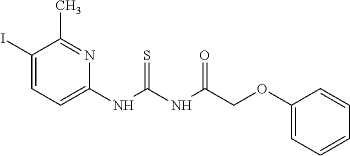 [SMILES: Cc1nc(NC(=S)NC(=O)COc2ccccc2)ccc1I] | 39 |

The results in Table 1 show that all the compounds tested showed at least a 10% level of melanin inhibition compared to a vehicle control. Compound NC-475 showed a greater than 50% inhibition, compounds S12, S15 and S17 showed levels of inhibition between 40 and 50%, compounds S13, S16 and S21 showed levels of inhibition of 30 to 40%, compounds S18, S19 and S20 showed levels of inhibition of 20 to 30% and S14 showed the lowest level of inhibition of 10%.

In the tyrosinase assay, a test compound at 10 μM that gave an inhibition of colour generation by more than 10% of the control value was considered to be a tyrosinase inhibitor. Kojic acid (5 μM) inhibited tyrosinase enzyme by 89%. 4-ethyl resorcinol (10 μM) inhibited tyrosinase activity by 29%. None of the compounds in Table 1 above were considered tyrosinase inhibitors in accordance with the above described assay.

Conclusions:

All the test compounds based on a core N-[[2-pyridinylamino]-thioxomethyl]-2-phenoxyacetamide structure have been found to be non-cytotoxic effective skin lightening compounds. Furthermore, none of the test compounds appears to operate through tyrosinase inhibition and can therefore be expected to have less of the safety issues which surround use of tyrosinase inhibiting skin lightening compounds.

1. A topical skin lightening composition comprising:
(a) A compound of formula (I): 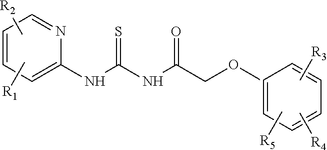
or a salt thereof;
wherein R₁ is —I or —Cl, R₂ is H or a methyl moiety, and, R₃, R₄ and R₅ may be independently selected from the group consisting of —H, -halide, and methyl, ethyl, propyl, iso-propyl, butyl, and t-butyl moieties; and
(b) a dermatogically acceptable vehicle.

2. A topical skin lightening composition according to claim 1, wherein R₁ and R₂ are at positions 5 and 6 on the pyridinyl moiety respectively.

3. A topical skin lightening composition according to claim 1, wherein R₃, R₄ and R₅ are —H.

4. A topical skin lightening composition according to claim 1, wherein R₃ and R₄ are —H.

5. A topical skin lightening composition according to claim 4, wherein R₅ is at the ortho or para position on the phenyl moiety.

6. A topical skin lightening composition according to claim 1, comprising 5 to 0.0005, preferably 2 to 0.005, most preferably 0.5 to 0.05% w/w compound of formula (I).

7. A topical skin lightening composition according to further comprising an ingredient selected from the group consisting of a fragrance, an additional skin lightening compound, a surfactant, an organic sunscreen, an inorganic sunscreen, an extender pigment, a preservative, an antiperspirant active, and a deodorant active.

8. A topical skin lightening composition according to claim 7, wherein the additional skin lightening compound is selected from the group consisting of niacinamide, a resorcinol, koijic acid, retinol, retinyl esters including retinyl palmitate, 12-hydroxystearic acid, lactic acid, ascorbic acid, glabradin, dioic acids including octadecenedioic acid and azelaic acid, resveratrol, ascorbyl phosphate, ascorbyl palmitate, acetyl glucosamine, calcium pantothenate, alpha arbutin, climbazole, pitera extract, soybean extract, undecylenoyl phenylalanine, aqueous extract of bearberry and mixtures thereof.

9. A topical skin lightening composition according to claim 8, wherein the resorcinol is selected from the group consisting of 4-ethyl resorcinol, 4-hexyl resorcinol and phenylethyl resorcinol.

10. A topical skin lightening composition according to claim 7, comprising an additional skin lightening compound which is a non-tyrosinase inhibiting skin lightening compound.

11. A topical skin lightening composition according to claim 1, comprising 0.0001-10, preferably 0.01-2, most preferably 0.1-1% w/w of additional skin lightening compound.

12. A topical skin lightening composition according to claim 1, comprising 0.01 to 15, preferably 0.1 to 10, most preferably 0.5 to 7.5% w/w an inorganic sunscreen and/or organic sunscreen.

13. A topical skin lightening composition according to claim 1 comprising 0.5 to 50, particularly from 5 to 30 and especially from 10 to 26% w/w an antiperspirant active.

14. A topical skin lightening composition according to claim 1, wherein the topical skin lightening composition is in the form of an emulsion.

15. A method of skin lightening comprising the step of applying to skin a topical skin lightening composition according to claim 1.

16. A method of skin lightening comprising the step of applying to skin a compound of formula (I): 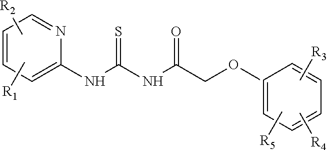
or a salt thereof;
wherein R₁ is —I or —Cl, R₂ is H or a methyl moiety, and, R₃, R₄ and R₅ may be independently selected from the group consisting of —H, -halide, and methyl, ethyl, propyl, iso-propyl, butyl, and t-butyl moieties.

17. A method of skin lightening according to claim 16, wherein R₁ and R₂ are at positions 5 and 6 on the pyridinyl moiety respectively

18. A method of skin lightening according to claim 16, wherein R₃, R₄ and R₅ are —H.

19. A method of skin lightening according to claim 16, wherein R₃ and R₄ are —H.

20. A method of skin lightening according to claim 19, wherein R₅ is at the ortho or para position on the phenyl moiety.

21. A method according to claim 20 wherein skin lightening is any one of the benefits selected from the group consisting of treating melasma, treating post-inflammatory hyperpigmentation, treating age spots, treating dark spots and treating uneven skin tone.

22-23. (canceled)
